(19) United States
(12) Patent Application Publication
(10) Pub. No.: US 2003/0180237 A1
(43) Pub. Date: Sep. 25, 2003

(54) Compositions for skin lightening and toning down pigment disorders, comprising creatinine and/or creatinine derivatives as active substances

(75) Inventors: Peter Lersch, Dinslaken (DE); Christian Weitemeyer, Essen (DE); Ute Wollenweber, Essen (DE)

Correspondence Address: SCULLY SCOTT MURPHY & PRESSER, PC, 400 GARDEN CITY PLAZA, GARDEN CITY, NY, 11530

(73) Assignee: Goldschmidt AG, Essen (DE)

(21) Appl. No.: 10/367,229

(22) Filed: Feb. 14, 2003

(30) Foreign Application Priority Data
Feb. 15, 2002 (DE) 02 003 483.1

(51) Int. Cl. A61K 7/135

(52) U.S. Cl. 424/62

(57) ABSTRACT

Compositions are provided for lightening skin and toning down pigment disorders which include, as active substances, at least one of the compounds of the general formula (I) and/or salts thereof

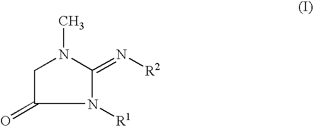

in which

R¹ is H, alkyl, hydroxyalkyl, or a carboxyalkyl radical having 2 to 30 carbon atoms; and

R² is H or a hydrocarbon radical having 1 to 30 carbon atoms which may be branched or unbranched and may or may not contain double bonds.

### DESCRIPTION

1. Field of the Invention

The present invention relates to compositions for skin lightening and toning down pigment disorders, and more particularly to compositions comprising creatinine and/or creatinine derivatives as active substances.

2. Background of the Invention

The color tone of human skin is determined by the amount of melanin present. Melanin is a brown-black pigment which is formed in the basal layer of the epidermis by special pigment-forming cells, referred to as the melanocytes. This pigmentation contributes somewhat to the UV protection of the skin because its absorption capacity can considerably weaken harmful UV radiation. Interestingly, people of differing skin color have a comparable number of melanocytes, only the formation rate of new melanin, its concentration and distribution are different.

UV irradiation induces the formation of melanin in special sections of the melanocytes, the so-called melanosomes. The melanin formed is transported into the keratinocytes, where it becomes visible as a brown skin color. The more melanin that is produced, the darker and more brown the skin is.

Uneven distribution of the melanocytes in sections of skin tissue leads to the undesired appearance of differing skin tones and local irregular hyperpigmentations, which manifest themselves, for example, in the form of pregnancy-related marks, age spots, freckles or other pigment disorders.

In chemical terms, melanin consists of polymeric indole-5,6-quinoids which are formed in a complex reaction cascade from the aromatic amino acid L-tyrosine. The reaction mechanism was explained previously by Raper and Mason and involves tyrosinases as a class of key enzymes.

Tyrosinase belongs to the family of type 3 copper proteins and is responsible for the hydroxylation of monophenols to give orthodiphenols. A detailed description is given, for example, in Angew. Chem. 2000, 112 (9), 1656-1660. Tyrosinase is activated by UV light, as a result of which it catalyzes the oxidative conversion of L-tyrosine to L-3,4-dihydroxyphenylalanine (L-Dopa). L-Dopa, in turn, is likewise oxidized by tyrosinase in further reaction steps to give dopaquinone and ultimately to give melanin. This mechanism occurs ubiquitously in nature, thus tyrosinases from fungi, plants and mammals are directly comparable with regard to their substrate specificity and action.

Undesired pigment disorders can be treated with depigmentation compositions, which are understood as meaning preparations for skin bleaching and/or skin lightening.

Particularly in Asiatic countries, many people feel the need to lighten their natural skin color since this corresponds to the ideals of beauty which prevail there.

In western countries, there is an increased interest in effectively evening out the appearance of irregularly pigmented sections of skin, which is often age-related, such as, for example, pregnancy-related marks or age spots.

There has hitherto been no lack of attempts to correct pigment disorders, and in the past, a large number of different substances have already been proposed which for their part intervene in various regulation mechanisms of pigment formation.

A targeted effect can be induced on skin tones and disorders by either breaking down the melanin present, or achieving a reduction in melanin formation.

For example, use was made previously of, inter alia, mercury and bismuth salts which irreversibly inhibit tyrosinase. However, due to the high toxicity of mercury and bismuth salts, such substances are no longer used in cosmetic compositions. The use of cell-toxic compounds, such as, for example, hydroquinone and derivatives thereof, which bring about direct destruction of the melanocytes and can only be applied to small areas of skin due to their harmful effect on the skin, is no longer approved in most countries either.

Most standard commercial skin-lightening compositions therefore usually comprise tyrosinase inhibitors of greater or lesser effectiveness. A number of substances have these properties. The palette of materials used therefore includes, in addition to highly diverse plant extracts, vitamin C and ascorbic acid derivatives, and also heterocyclic compounds, such as, for example, pyranone derivatives.

An overview of the topic and the substances used is given, inter alia, in Cosmetics & Toiletries 1995, 110 (10), 51-56. However, upon closer analysis, these substances do not entirely meet the requirements placed on them.

In this connection, the use of kojic acid (5-hydroxy-2-hydroxymethyl-4-pyranone), for example, is known. GB-A-826244 describes methods for the fermentative preparation of kojic acid, which is obtained in a multistage process from Aspergillus cultures. Examples of the use of the substance and its derivatives in skin-lightening compositions can be found widely in the patent literature. By way of representation, reference may be made here to EP-A-0 308 543.

The activity mechanism of kojic acid and its derivatives is thought to be based on a chelation of the catalytically active central copper atom in tyrosinase. However, for the incorporation into cosmetic preparations the instability of kojic acid in aqueous solutions has proven to be quite a disadvantage since, in addition to a high loss in effectiveness, it can also contribute to stability problems of the formulation.

There is therefore still a need for active ingredients for cosmetic and dermatological formulations which are able to bleach or lighten the natural skin color and/or effectively balance out the appearance of irregularly pigmented areas of skin, such as, for example, pregnancy-related marks or age spots.

Preferably, such an active ingredient should bring about a significant effect even in low use concentrations. Moreover, such an active ingredient should be nontoxic, preferably be natural in origin, be very well tolerated by the skin, have a high compatibility with other ingredients and be able to be incorporated into skin-treatment compositions without problems.

It is particularly desirable if this active ingredient can additionally be prepared in a simple and cost-effective manner and be produced in a form which can be purified easily and thus satisfies the high purity requirements placed on cosmetics and dermatological active ingredients.

It is therefore an object of the present invention to provide such active ingredients which have the ability, in cosmetic and dermatological formulations, to lighten the natural skin color and/or to effectively even out the appearance of irregularly pigmented areas of skin, such as, for example, pregnancy-related marks or age spots.

### SUMMARY OF THE INVENTION

Surprisingly, it has now been found that creatinine and/or creatinine derivatives in preparations for the treatment and after-treatment of the skin satisfy all of these desired criteria.

The present invention therefore provides compositions for lightening the skin and toning down pigment disorders which comprise, as active substances, at least one of the compounds of the general formula (I) and/or salts thereof

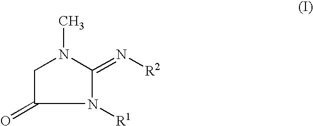

in which

R¹ is H, alkyl, hydroxyalkyl, or a carboxyalkyl radical having 2 to 30 carbon atoms; and

R² is H or a hydrocarbon radical having 1 to 30 carbon atoms which may be branched or unbranched and may or may not contain double bonds.

In one embodiment of the present invention, R¹ and/or R² have 2 to 20 carbon atoms.

The present invention further provides cosmetic or dermatological formulations for lightening the skin and toning down pigment disorders, comprising at least one of the compounds of the general formula (I) and/or salts thereof. Specifically, the cosmetic or dermatological formulation of the present invention includes 0.05 to 10 parts by weight of at least one of the compounds of the general formula (I), and 1 to 10 parts by weight of at least one surfactant selected from the group consisting of nonionic, amphoteric, zwitterionic, and ionic surfactants. The formulation may optionally include 2 to 10 parts by weight of vegetable, mineral, or ester oils, 1 to 5 parts by weight of bodying agents, 0.5 to 5.0 parts by weight of fragrances, dyes, plant extracts, preservatives, and ad 100% with water.

### DETAILED DESCRIPTION OF THE INVENTION

Creatinine (CAS No. 60-27-5) is the trivial name for 2-imino-N-methylhydantoin, a cyclic condensation product which can be obtained by intramolecular elimination of water from creatine (formula II).

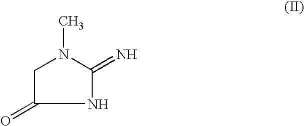

Creatinine occurs naturally within the body and is used in medicine as a biological marker. Further information on this topic is given, inter alia, in D. W. Cockcroft and M. H. Gault in Prediction of creatinine clearance from serum creatinine, Nephron. 1976 (16)31-41.

Creatinine is regularly found in urine and is formed as a result of an amidino group transfer from arginine to glycine. It is also present in perspiration and a further role is attributed to the substance as a constituent of the natural moisturizing factor of skin.

A few patent applications have recently already been published in which the use of creatinine in skin care products is described.

For example, WO-A-00/15187 (SKW Trostberg) discloses the use of creatine as humectant in cosmetic preparations. The use of creatine and/or suitable derivatives is disclosed to correct symptoms of dry skin, such as cracks and flaking, with lasting effect.

JP-0247866 (Lion Corporation) describes skin cosmetics which comprise creatine and/or creatinine in combination with a further pharmaceutical active ingredient and/or a bioactive substance and are disclosed to have an improved care effect. Within the framework of a general listing, the use of creatinine in combination with skin whitening agents, such as ellagic acid, hydroquinone, arbutin, kojic acid and other materials, is actually described. The effective skin-lightening effect is attributed solely to the whitening agents. The effect of creatinine and its derivatives in their own right, however, have not been recognized in this publication and therefore no indication of such properties can be deduced from this specification.

A further application is WO-A-01/00203 (Avicena), which discloses the use of creatine and creatine derivatives as antioxidants and for the regeneration of stressed skin.

The literature therefore suggests nothing about the use of creatinine or derivatives thereof in skin-treatment compositions for the purposes of skin lightening or the bleaching of irregularly pigmented areas of skin.

For the purposes of the present invention, creatinine has proved particularly suitable and is therefore preferred. However, creatinine derivatives, such as its salts with inorganic acids, such as, for example, phosphoric acid, preferably organic mono- or polybasic acids, such as, for example, acetic acid, glycolic acid, lactic acid, citric acid, malic acid, salicylic acid or sorbic acid and mixtures thereof, or such as creatinine pyruvate are also very suitable. In this respect, for the purposes of the present invention, it is also possible to use suitable creatinine derivatives in mixtures with one another.

The creatinine derivatives of the general formula (I) can be prepared by customary esterification, amidation and alkylation or addition processes or in accordance with these processes. In this respect, reference may be made to the relevant specialist literature, such as, for example, Houben-Weyl, Methoden der Organischen Chemie [Methods of organic chemistry] 4TH EDITION, Supplementary Series, Volume E4 CARBONIC ACID DERIVATIVES (1983) and E5 CARBOXYLIC ACID, DERIVATIVES (1985), and also to the reports, published annually, relating to “Guanidino Compounds in Biology and Medicine” (Eds. P. P. DeDeyn, B. Marescau, V. Stalon, J. A. Qureshi), John Libbey and Co. Ltd., London.

Creatinine itself exists in an aqueous solution in an equilibrium with creatine. It is possible to shift the equilibrium depending on the pH and temperature. Thus, for the cosmetic or dermatological formulations it is possible to use both creatine and creatine/creatinine mixtures, provided it is ensured, by observing corresponding storage and/or application conditions, that an adequately effective content of creatinine is present during the application.

Cosmetic or dermatological preparations for depigmentation according to the present invention are understood primarily as meaning those compositions which are applied to the facial skin and/or other hyperpigmented parts of the body.

These compositions are the customarily used cosmetic or dermatological formulations, which are generally in the form of aqueous alcoholic solutions, creams, emulsions, lotions, gels, aerosol spray or foam, non aerosol spray or foam, and in which the compounds of the general formula (I) are utilized. The compositions can, accordingly, be tailored to the use, and the composition may also comprise further customary constituents which serve for the treatment, care, cleansing and protection of the skin, such as, for example, skin-cosmetic active ingredients, such as, for example, creatine, ceramides, pseudoceramides, protein hydrolysates of vegetable or animal origin based on keratin, collagen, elastin, wheat, rice, soybean, milk, silk, corn, amino acids and amino acid derivatives, polyaspartic acid (derivatives), anti-inflammatory active ingredients, antimicrobial active ingredients, customary antioxidants, vitamins, dexpanthenol, lactic acid, pyrrolidone carboxylic acid, bisabolol, and plant, yeast and algae extracts.

The combination with customarily used organic or inorganic UV filter substances is regarded as being particularly advantageous since during the use of the skin-bleaching formulations, the formation of new melanin in the skin is effectively suppressed and, as a result, the natural protective function of the melanin is reduced, and secondly, by avoiding exposure to sunlight, the production of new melanin is prevented.

In addition, other cosmetic auxiliaries and additives which are customary in such preparations may also be present. Such auxiliaries include, for example, solubility enhancers, such as ethanol, isopropanol, ethylene glycol, propylene glycol, glycerol and diethylene glycol. Other components include cosmetic oils of vegetable and synthetic origin, emollients, fats, waxes, refatting agents, emulsifiers,. thickeners, anionic, zwitterionic, amphoteric and nonionic surfactants as well as fragrances and preservatives.

Finally, the formulations according to the present invention can also comprise complexing agents, such as, for example EDTA, NTA, β-alaninediacetic acid and phosphonic acid, dyes for coloring the cosmetic preparation, opacifiers, such as latex, styrene/PVP and styrene-acrylamide copolymers, pearlizing agents, such as ethylene glycol mono- and distearate and PEG-3 distearate, pigments, light protection agents, thickeners or propellants.

Typical guideline formulations for skin-treatment compositions belong to the known prior art and are given, for example, in the brochures from the manufacturers of the respective basic substances and active ingredients. An informative source of such formulations is, for example, the Kosmetik-Jahrbuch [Cosmetics year book], which is published annually, (publisher: B. Ziolkowsky, Verlag für Chemische Industrie).

These existing formulations can usually be adopted without change. Where necessary, for adaptation and optimization, the desired modifications can, however, be undertaken by simple experiments without complications.

Creatinine and its derivatives can generally be present in a concentration of from 0.05 to 10.0% by weight, preferably in a concentration of from 0.2 to 5.0% by weight.

One example of an entirely customary recipe for a skin cream formulation is given below. This is composed of:

Basic recipe: Skin cream

Compound of the present invention: 0.05 to 10 parts by weight;

Glycerol monodistearate: 2 to 10 parts by weight;

Cetyl alcohol: 1 to 4 parts by weight;

Paraffin oil 3.5° E: 4 to 12 parts by weight;

Glycerol: 1 to 5 parts by weight;

Demineralized water: ad 100;

Preservative: n.d.

The cosmetic preparations according to the present invention for the lightening treatment of skin have a pH of from 3 to 7 and therefore preferably comprise a water-soluble acid or buffer mixture suitable for stabilizing this pH. Suitable acids are, in particular, the low molecular weight organic acids, such as, for example, acetic acid, glycolic acid, lactic acid, citric acid, malic acid, salicylic acid or sorbic acid and mixtures of these acids with their alkali metal salts.

The formulations according to the present invention are prepared in the customary manner, whereby the creatinine and derivatives thereof are preferably dissolved in the aqueous phase of the formulation. The pH is adjusted, preferably at the end, by adding the acid and/or buffer mixture intended for this purpose. To improve the solubility of the creatinine and its derivative, the preparation according to the present invention can be gently heated prior to application to the skin.

The examples below serve to illustrate the subject-matter of the present invention in more detail:

### EXAMPLE 1

Tyrosinase inhibition by creatinine

The inhibition of fungi tyrosinase was determined as a function of different concentrations of creatinine (0.05 to 1.7% by volume) by reference to the enzymatic reaction of L-Dopa to dopachrome. The absorption maximum of the dopachrome is at a wavelength of 475 nm. The tyrosinase inhibition is calculated in accordance with the following equation:

Inhibition [%]={100−[(Eₛₐₘₚₗₑ* 100)/E_{reference}]} (1)

each measurement was carried out in duplicate, with parallel mixtures. The variation of the method is ±10%.

Chemicals used:

L-3,4-Dihydroxyphenylalanine (L-DOPA) (Sigma)
Tyrosinase, 25,000 units (Sigma)

Solutions used:

25 mM acetate buffer (pH 4.9): dilute from 0.2 M acetate buffer (pH 4.9) (Sigma)

Phosphate buffer (pH 7): Titrosol (Merck)

20 mM L-DOPA (in acetate buffer)

Tyrosinase stock solution: 40 U/ml of phosphate buffer

Stock solution of creatinine: 0.45 M in phosphate buffer

The DOPA solution and the tyrosinase stock solution were prepared only prior to the start of the experiment. The L-DOPA solutions should be stored in a dark place and in vessels which can be sealed tightly.

Implementation:

500 μL of DOPA stock solution

400 μL of creatinine solution or phosphate buffer (reference)

100 μL of tyrosinase stock solution

Following the addition of the enzyme, the samples were thoroughly mixed using a “Reax Top” (Heidolph). Following an incubation period of 15 min at room temperature, the absorption at 475 nm was determined. The measurement was carried out using a “Helios Beta” spectrophotometer from Unicam.

Result:

Table 1 summarizes the tyrosinase inhibition as a function of creatinine concentration.

TABLE 1

| Concentration (% by volume) | Tyrosinase inhibition (%) |
|---|---|
| 0.00 | 0.00 |
| 0.05 | 1.77 |
| 0.30 | 9.97 |
| 0.60 | 14.21 |
| 0.90 | 20.75 |
| 1.10 | 24.81 |
| 1.40 | 28.15 |
| 1.70 | 32.18 |

The tyrosinase-inhibiting properties characterize creatinine as a moderate skin lightener, and advantageous for use in cosmetics with a depigmentating action.

### EXAMPLE 2

In vitro skin model test

The skin-lightening effect was validated on an in vitro skin model, the MelanoDerm™ from MatTek. This model is very similar in structure and function to the natural skin and has the cell types relevant in the epidermis including the melanocytes, which are responsible for the synthesis of the main pigment melanin. Further information can be found, for example, in M. K. King et al. in Proceedings of Society of Cosmetic Chemists Annual Scientific Meeting and Technology Showcase 1998, 35-36.

Creatinine, kojic acid dipalmitate as positive standard and water as negative control were applied every 48 hours over a period of 21 days to the in vitro skin model.

Result:

The melanin produced by the melanocytes was determined quantitatively on the 17th and 21 st day. Table 2 summarizes the results.

TABLE 2

Melanin (μg)

| | Day 17 | Day 21 |
|---|---|---|
| Creatinine | 94.5 | 12.67 |
| Kojic dipalmitate | 102.5 | 82.67 |
| Water | 205.0 | 162.17 |

Table 2 demonstrates that creatinine is twice as effective upon prolonged application as the kojic acid dipalmitate used as positive standard.

Formulation example according to the present invention:

| Skin-lightening cream | % by wt. |
|---|---|
| Phase A | |
| ABIL ® Care 85 | 1.5 |
| (Bis-PEG/PPG-16/16 PEG/PPG-16/16 dimethicone; | |
| caprylic/capric triglycerides) | |
| TEGINACID ® C (Ceteareth-25) | 0.5 |
| TEGIN ® M (Glyceryl stearate) | 2.0 |
| TEGO ® Alkanol 1618 (Cetearyl alcohol) | 6.0 |
| Cyclomethicone | 5.0 |
| Phase B | |
| Compound of formula (I) R¹, R² = H (creatinine) | 0.5 |
| Glycerol | 3.0 |
| Water | ad 100 |
| Phase C | |
| TEGO ® Carbomer 134 (carbomer) | 0.15 |
| TEGOSOFT ® (Ethylhexyl palmitate) | 0.6 |
| Phase D | |
| Sodium hydroxide (10% in water) | q.s. |
| Preservative, perfume | q.s. |
| Preparation: | |

1. Heat phase A and B to about 75° C.
2. Add phase A with stirring to phase B.¹⁾
3. Homogenize.
4. Cool, with stirring, to about 60° C. and then add phase C.
5. Briefly homogenize.
6. Cool with stirring and add phase D below 40° C.
¹If phase A is introduced first, phase B is added without stirring.

Three people applied the cream described above twice daily over a period of 2 months for the treatment of pigment spots on the upper arm. After just 4 weeks, a clearly visible lightening could be detected and after two months a considerably visible improvement in the appearance of the skin, i.e., a significant reduction in local pigment disorders was evident. Skin irritations were not observed throughout the entire treatment period.

While the present invention has been particularly shown and described with respect to preferred embodiments thereof, it will be understood by those skilled in the art that the foregoing and other changes in forms and details may be made without departing from the spirit and scope of the present invention. It is therefore intended that the present invention is not limited to the exact forms and details described and illustrated, but fall within the scope of the appended claims.

What we claim is:

1. A composition for lightening skin and toning down pigment disorders, which comprises, as active substances, at least one compound of formula (I) and/or salts thereof 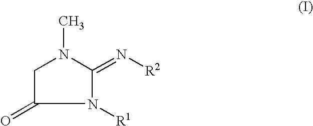
in which
R¹ is H, alkyl, hydroxyalkyl, or a carboxyalkyl radical having 2 to 30 carbon atoms; and
R² is H or a hydrocarbon radical having 1 to 30 carbon atoms.

2. The composition of claim 1 wherein R¹ and/or R² have 2 to 20 carbon atoms.

3. The composition of claim 1 wherein R¹ and/or R² are hydrogen.

4. The composition of claim 1 wherein 0.05 to 10.0% by weight of at least one of the compounds of the general formula (I) is present as the active substance.

5. The composition of claim 1 wherein 0.2 to 5.0 % by weight of at least one of the compounds of the general formula (I) is present as the active substance.

6. The composition of claim 1 wherein R² is a branched hydrocarbon radical.

7. The composition of claim 1 wherein R² is a hydrocarbon radical that contains double bonds.

8. A cosmetic or dermatological formulation for lightening skin and toning down pigment disorders, comprising at least one compound of the general formula (I) and/or salts thereof 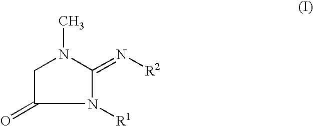
in which
R^{1 -}is H, alkyl, hydroxyalkyl, or a carboxyalkyl radical having 2 to 30 carbon atoms; and
R² is H or a hydrocarbon radical having 1 to 30 carbon atoms.

9. The cosmetic or dermatological formulation of claim 8 comprising 0.05 to 10.0% by weight of at least one of the compounds of the general formula (I) and/or salts thereof.

10. The cosmetic or dermatological formulation of claim 8 comprising 0.2 to 5.0% by weight of at least one of the compounds of the general formula (I) and/or salts thereof.

11. The cosmetic or dermatological formulation of claim 8 wherein R¹ and R²=H.

12. The cosmetic or dermatological formulation of claim 8 comprising
a) 0.05 to 10 parts by weight of at least one of the compounds of the general formula (I), and
b) 1 to 10 parts by weight of at least one surfactant selected from the group consisting of nonionic, amphoteric, zwitterionic, and ionic surfactants.

13. The cosmetic or dermatological formulation of claim 12 further comprising
c) 2 to 10 parts by weight of vegetable, mineral, or ester oils,
d) 1 to 5 parts by weight of bodying agents,
e) 0.5 to 5.0 parts by weight of fragrances, dyes, plant extracts, preservatives, and
f) ad 100% with water.

14. The cosmetic or dermatological formulation of claim 8 wherein R² is a branched hydrocarbon radical.

15. The cosmetic or dermatological formulation of claim 8 wherein R² is a hydrocarbon radical that contains double bonds.
